(19) United States
(12) Patent Application Publication
(10) Pub. No.: US 2015/0128960 A1
(43) Pub. Date: May 14, 2015

(54) HEAD AND FACIAL PROTECTIVE DEVICE

(71) Applicant: Kristin C. Bauman, Traverse City, MI (US)

(72) Inventor: Kristin C. Bauman, Traverse City, MI (US)

(21) Appl. No.: 14/078,004

(22) Filed: Nov. 12, 2013

(51) Int. Cl. A61F 5/37

(52) U.S. Cl. CPC A61F 5/3707
USPC 128/857

(57) ABSTRACT

The present invention provides a device for protecting the face and/or head of an individual receiving medical treatment in a supine position. The protective device includes a central member having an exterior surface and an interior surface. One or more apertures may extend between the interior and exterior surface of the central member to expose the individual's eyes, nose, and/or mouth. Affixed on generally opposite ends of the central member are side portions that extend downward from the central member. The central member and side portions define a cavity and an opening configured to be positioned over the head of an individual receiving medical treatment in a supine position with the side portions supported on a support surface adjacent the individual's head. The protective device directs forces applied to the top surface of the central member away from the individual's head through the side portions to the supporting surface.

### FIELD OF THE INVENTION

The present invention is directed to a protective device and, more particularly, to a method and apparatus for protecting the head and/or face of a patient lying in the supine position.

### BACKGROUND OF THE INVENTION

Protecting a patient's face during surgery is an important concern. Unintentional pressure on the ocular structures can cause severe damage and even blindness. Uneven pressure on the face can lead to decreased blood flow, potentially resulting in nerve damage. Compression injuries to the forehead, nose, ears, cheeks, and mouth can also occur. Once a patient undergoing a surgical procedure is fully anesthetized under a general anesthetic, the patient is no longer able to move in response to an uncomfortable or painful stimulus. Therefore, constant vigilance by the anesthesia provider is necessary to protect the vital facial structures.

### SUMMARY OF THE INVENTION

The present invention provides a device and method for protecting the face and/or head of an individual receiving medical treatment in a face up, or supine, position.

According to an aspect of the present invention, a protective device is provided that includes a central member having an exterior surface and an interior surface. Affixed on generally opposite ends of the central member are side portions that extend downward from the central member. The central member and side portions define a cavity and an opening to the cavity with the protective device configured to be positioned over the head of an individual receiving medical treatment in the supine position with the side portions supported on a support surface adjacent the individual's head. The protective device directs forces applied to the top surface of the central member away from the individual's head through the side portions to the supporting surface. One or more apertures may extend between the interior and exterior surface of the central member to expose the individual's eyes, nose, and/or mouth when the protective device is positioned over the individual's head. The protective device may also include one or more retention members for securing or holding items such as an endotracheal tube, anesthesia circuit or the like to the protective device.

According to another aspect of the present invention, a method of protecting the face and/or head of an individual receiving medical treatment while lying in the supine position comprises providing a protective device having a central member with an exterior surface and an interior surface and a pair of side portions extending downward from generally opposite ends of the central member. The central member and side portions define an opening and a cavity with the protective device configured to be positioned over the individual's head with the side portions supported on a support surface adjacent the individual's head.

These and other objects, advantages, purposes and features of the present invention will become more apparent upon review of the following specification in conjunction with the drawings.

### BRIEF DESCRIPTION OF THE DRAWINGS

FIG. 1 is a top perspective view of a facial and head protective device in accordance with the present invention shown in use on a patient;

FIG. 2 is a front-right perspective view of the protective device of FIG. 1 shown without the patient;

FIG. 3 is a top plan view of the protective device of FIG. 2;

FIG. 4 is a bottom plan view of the protective device of FIG. 2;

FIG. 5 is a left elevation view of the protective device of FIG. 2;

FIG. 6 is a right elevation view of the protective device of FIG. 2;

FIG. 7 is a top end elevation view of the protective device of FIG. 2; and

FIG. 8 is a bottom end elevation view of the protective device of FIG. 2.

### DESCRIPTION OF THE PREFERRED EMBODIMENTS

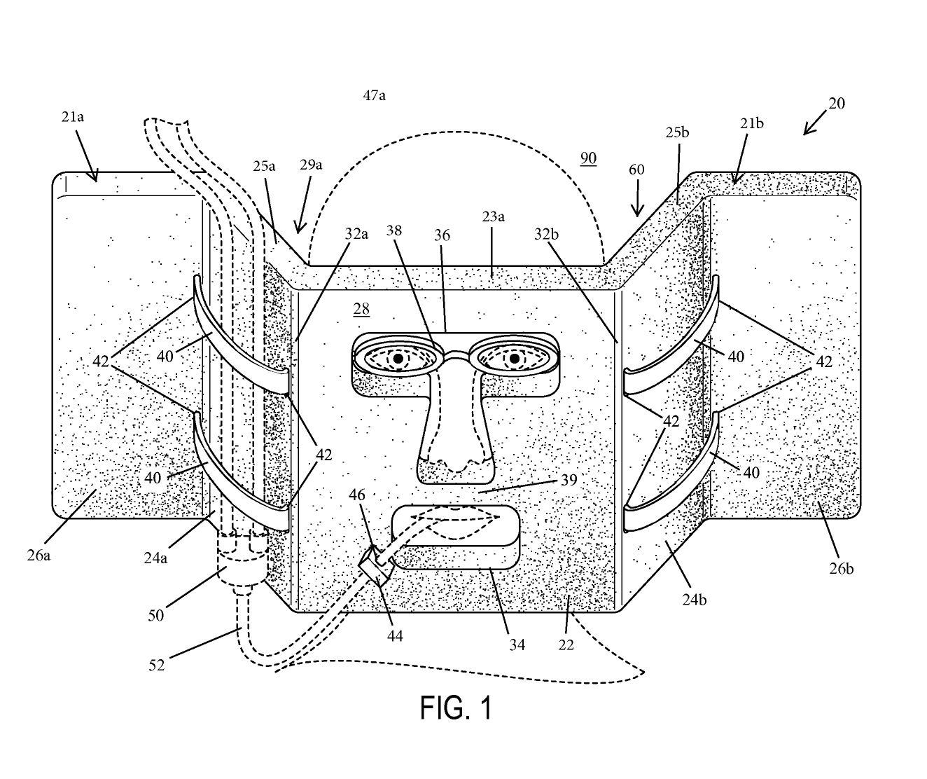

The present invention will now be described with reference to the accompanying figures, wherein the numbered elements in the following written description correspond to like-numbered elements in the figures. Referring to FIG. 1, a facial and head protective device 20 comprises a face portion or a central member 22 and a pair of side legs or portions 21a, 21b extending from opposite sides of central member 22, with member 22 also including apertures 34 and 36. Together, side portions 21a, 21b and member 22 define a recessed area or cavity 60 for receiving the head of a patient lying in a supine position when protective device 20 is placed over or about the patient's head and the distal ends of side portions 21a, 21b contact a bed, gurney, pillow, or other support surface 90 on each side of the patient's head. When so arranged apertures 34 and 36 are aligned with the patient's eyes, nose and mouth. Protective device 20 is adapted to direct forces applied thereto, such as from medical equipment or the like, to the support surface 90 to thereby protect the patient. Moreover, in the illustrated embodiment protective device 20 comprises a soft, foam-type padding that cushions the patient's face, protects pressure points, and may engage the patient's facial structures to aid in preventing shifting of protective device 20 during medical procedures, such as surgeries and the like.

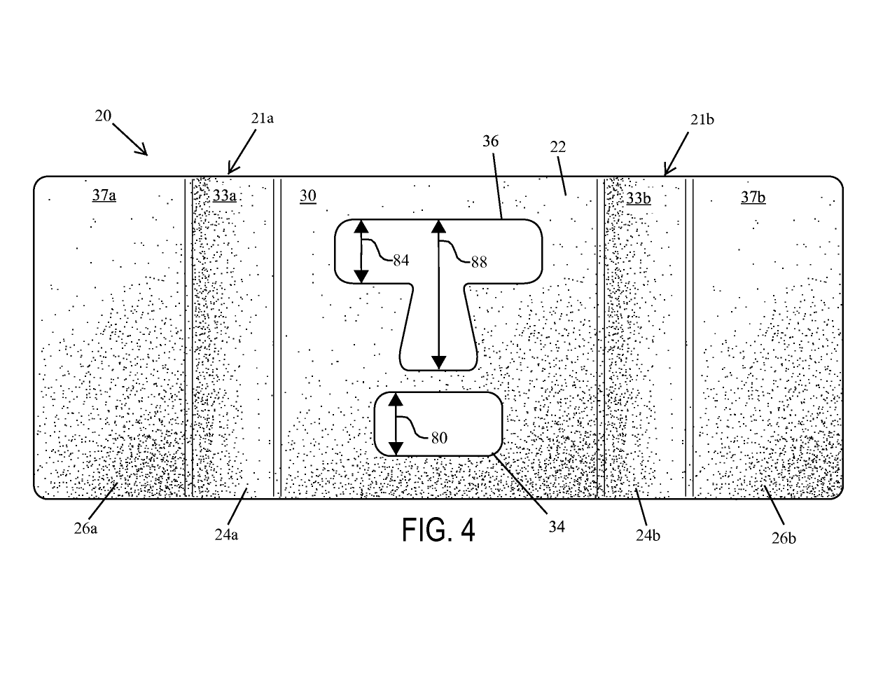

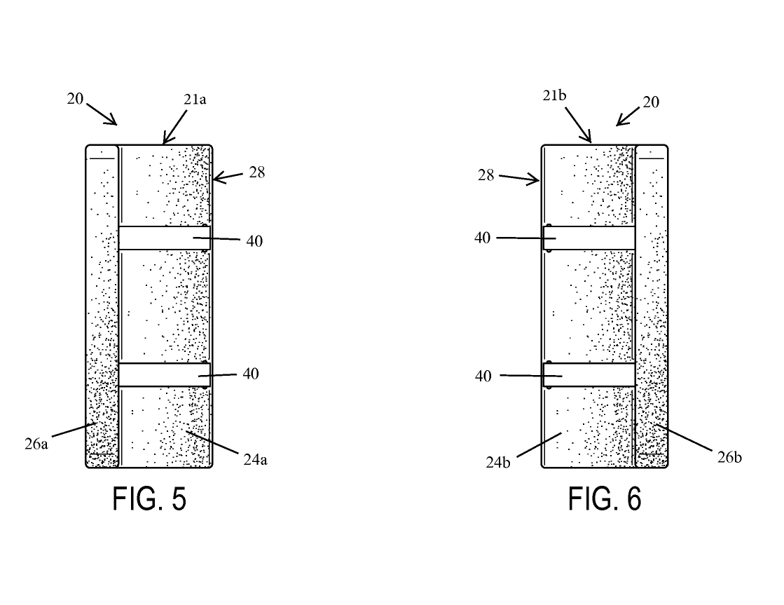

In the illustrated embodiment the side legs 21a, 21b of protective device 20 include side members 24a and 24b and corresponding base elements or members 26a and 26b, respectively. Side members 24a and 24b extend downward from generally opposite sides 32a and 32b of member 22. Base members 26a and 26b extend outward and away from side members 24a and 24b, respectively, in a plane generally parallel to central member 22. Central member 22 has both a top surface 28 and a bottom surface 30 (FIG. 4). Contact between base members 26a and 26b and support surface 90 further help in preventing shifting of protective device 20 and also allow forces applied to top surface 28 of member 22 to be directed away from the patient's face and head through side members 24a and 24b and base members 26a and 26b to support surface 90. Additionally, central member 22, side members 24a and 24b, and base members 26a and 26b help prevent the patient's head from falling sideways, potentially decreasing the blood flow through the neck veins and arteries and leading to nerve damage.

Top surface 28 of member 22 is generally flat, providing a surface on which to rest surgical instruments or devices. Protective device 20 is sized whereby bottom surface 30 makes light contact with or is just above the patient's face. Member 22 may include one or more openings or apertures for various facial structures. In the illustrated embodiment, central member 22 has a generally rectangular aperture 34 for the mouth and a generally T-shaped aperture 36 for the nose and eyes, with the apertures 34, 36 being separated by a bridge portion 39 that is located over the individual's upper lip when protective device 20 is positioned about the patient's head. Apertures for other structures such as the patient's ears are also envisioned. Apertures 34 and 36 not only permit bottom surface 30 of central member 22 to make light contact with the patient's face, they also provide easy access to the eyes, nose, and mouth during surgery. Optionally, protective device 20 may further include a transparent eye covering or goggles 38 built in or attached to aperture 36 to offer additional protection for the patient's eyes while still allowing the anesthesia provider to easily view and monitor the patient's condition throughout the procedure.

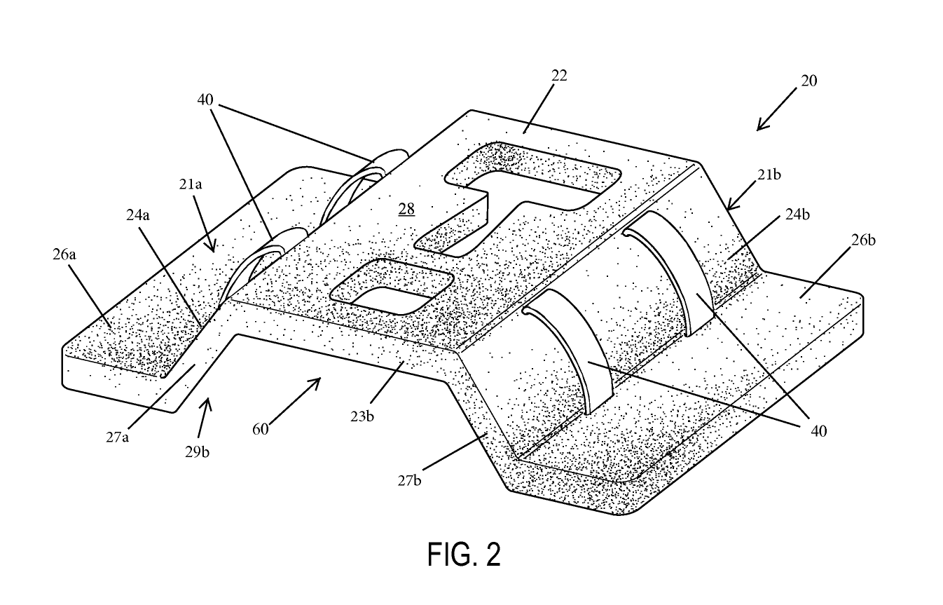

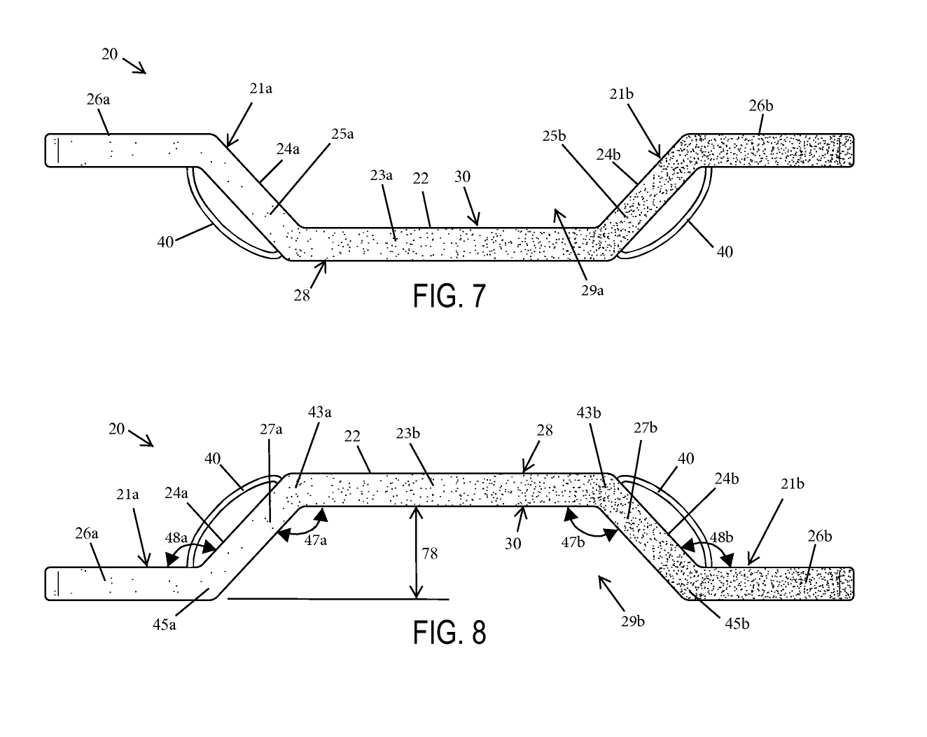

With reference to FIGS. 1 and 7, member 22 includes upper edge 23a and side members 24a, 24b include upper edges 25a, 25b, respectively. Similarly, with reference to FIGS. 2 and 8, member 22 includes a lower edge 23b and side members 24a, 24b include lower edges 27a, 27b, respectively. Upper edges 23a, 25a and 25b define an upper opening 29a to cavity 60, and lower edges 27a, 23b and 27b define a lower opening 29b to cavity 60. As understood from FIG. 1, when in use upper opening 29a exposes the top of the individual's head, with lower opening 29b being disposed about the individual's neck.

On each side of protective device 20 may be one or more retaining members 40, which may be used to attach various devices and instruments such as an anesthesia circuit 50 or the like. Securing anesthesia circuit 50 to protective device 20 helps to stabilize circuit 50 and protect against unwanted kinking in the tubing, which can lead to inaccurate readings for the anesthesia provider. Retention members 40 on both sides of protective device 20 allow the anesthesia provider to easily secure anesthesia circuit 50 to either the left or right side of the patient. In the illustrated embodiment retention members 40 are constructed as hook and loop fastening mechanisms or straps that are attached to protective device 20 at side members 24a and 24b and base members 26a and 26b through a plurality of slots 42. However, it is envisioned that devices and instruments may be attached to protective device 20 at any number of locations and through any number of means such as, for example, tape, adhesive, or the like. One skilled in the art will recognize and appreciate the various ways devices and instruments may be attached to protective device 20.

Protective device 20 is shown in FIG. 1 with an optional protrusion or holding member 44 affixed to member 22, with holding member 44 being positioned near the mouth aperture 34 for holding or retaining an endotracheal tube 52 inserted into the patient's mouth. Optionally, holding member 44 may be releasably attached to central member 22 such as, for example, through a hook and loop attachment so that holding member 44 may be moved by the anesthesia provider to either side of the patient's mouth and reattached. As shown, holding member 44 has a through-hole 46 that serves as the attachment mechanism for endotracheal tube 52. Optionally, the attachment mechanism for endotracheal tube 52 may take any number of forms, such as a press-fit slot, while remaining within the spirit and scope of the present invention.

In operation, protective device 20 is aligned so apertures 34 and 36 are generally over the appropriate facial structures of the patient. Protective device 20 is then placed over the patient's face so base members 26a and 26b make contact with support surface 90 on each side of the patient's head. Optionally, if protective device 20 is so equipped, transparent goggles 38 may be placed over the patient's eyes and anesthesia circuit 50 and endotracheal tube 52 may be secured to either the left or right side of protective device 20 through the use of retaining members 40 and/or holding member 44.

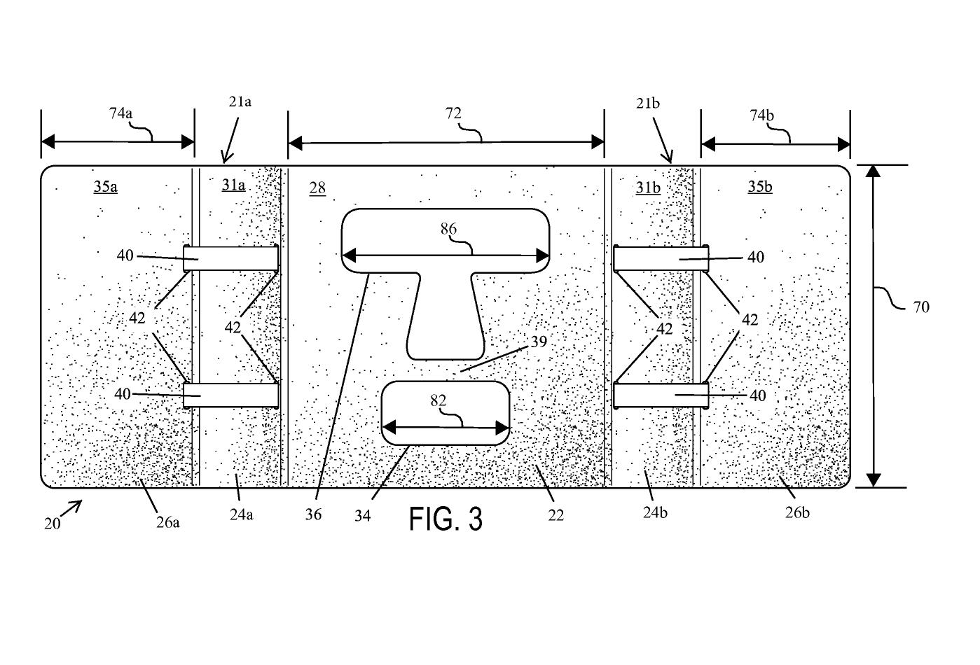

In the embodiment shown, central member 22, side members 24a and 24b, and base members 26a and 26b are each generally rectangular in shape. Side members 24a, 24b each have planar top surfaces 31a, 31b (FIG. 3) and bottom surfaces 33a, 33b (FIG. 4). Base members 24a, 24b each have planar top surfaces 35a, 35b (FIG. 3) and bottom surfaces 37a, 37b (FIG. 4). Side members 24a, 24b are connected to member 22 at angled joints 43a and 43b (FIG. 8), respectively. Side members 24a, 24b are also connected to base members 26a, 26b at angled joints 45a and 45b (FIG. 8), respectively. In the illustrated embodiment the internal surfaces of member 22 and side members 24a, 24b at joints 43a, 43b define obtuse angles 47a and 47b (FIG. 8), respectively, and the exterior surfaces of side members 24a, 24b relative to base members 26a, 26b at joints 45a, 45b also define obtuse angles 48a and 48b (FIG. 8), respectively. Also in the illustrated embodiment, the overall length 70 (FIG. 3) of protective device 20 is approximately 10 inches, with member 22 having a width 72 (FIG. 3) of approximately 10 inches. Base members 26a and 26b have widths 74a and 74b (FIG. 3), respectively, measuring about 5 inches. Side members 24a and 24b extend downward from opposite sides of central member 22 to base members 26a and 26b defining cavity 60 having a depth 78 (FIG. 8) of approximately 6 inches. It should be appreciated, however, that alternative shapes and sizes may be employed. For example, the size of central member 22 and depth 78 of cavity 60 may vary to accommodate the different physical sizes of patients' heads.

Referring now to FIGS. 3 and 4, central member 22 is shown having two apertures 34 and 36. Aperture 34 is positioned to generally align with the patient's mouth and aperture 36 is positioned to generally align with the patient's nose and eyes. Aperture 34 is essentially rectangular in shape having a height 80 of approximately 2 inches and a width 82 of approximately 4 inches. Aperture 36 is generally T-shaped with the opening for the eyes having a height 84 of about 2 inches and a width 86 of about 6.5 inches. The opening for the nose has a height 88 of approximately 4.3 inches. Such sizes are selected to accommodate variations in facial dimensions. However, it should be recognized that alternative shapes, sizes, and number of apertures may be employed.

In the illustrated embodiment, protective device 20 is formed by a molding process with central member 22, side members 24a and 24b, and base members 26a and 26b comprising a unitary piece of a soft, solid foam material to provide cushioned protection for the face and cranial region of the patient. Such material is light weight and easy to cut in order to gain access to the face. Additionally, such material is generally inexpensive, so protective device 20 may be disposed of after each individual use, avoiding the need to sterilize device 20 between uses. It should be appreciated, however, that protective device 20 may be formed from foam material of varying degrees of rigidity or may be constructed from any suitable light weight material, such as a plastic or polymeric material or the like, capable of providing protection for the face and cranial region of the patient. Further, as an alternative to single piece construction, protective device 20 may optionally be assembled from two or more pieces fixedly attached to each other.

In the illustrated embodiment, base elements 26a and 26b extend outward and away from side members 24a and 26b, respectively, forming a planar surface generally parallel to central member 22. It is envisioned, however, that base elements do not have to extend outward from side members 24a and 24b, but rather may be the bottom ends of side members 24a and 24b. The base elements still lie in generally in a plane parallel to central member 22 and make contact with support surface 90 to allow forces applied to top surface 28 of central member 22 to be directed to support surface 90.

The apparatus and method of the present invention provide protection for a patient undergoing surgery in the supine position by allowing forces to be directed away from the sensitive areas of the face and cranial region. The apparatus and method of the present invention also help to keep the patient's head from falling to the side, further reducing the risks of decreased blood flow and possible nerve damage. The apparatus and method, therefore, provide increased protection over conventional practices.

Changes and modifications to the specifically described embodiments may be carried out without departing from the principles of the present invention, which is intended to be limited only by the scope of the appended claims as interpreted according to the principles of patent law including the doctrine of equivalents.

1. A protective device for protecting the face and/or head of an individual receiving medical treatment, said protective device comprising:
a central member having an exterior surface and an interior surface; and
a pair of opposed side portions affixed to said central member and extending downwardly there from relative to a plane defined by said central member;
said central member and said side portions defining a cavity and an opening to said cavity with said protective device configured to being positioned over the head of an individual receiving medical treatment in a supine position with said side portions being supported on a support surface adjacent the individual's head and said cavity being disposed over the individual's head with the individual's neck extending into said opening and said interior surface being aligned with the individual's face.

2. The protective device of claim 1, wherein said central member includes an aperture extending between said exterior surface and said interior surface.

3. The protective device of claim 2, wherein said aperture is configured to expose an individual's eyes, nose, and/or mouth when said protective device is positioned over the individual's head.

4. The protective device of claim 2, wherein said aperture comprises a first aperture and said central member further includes a second aperture extending between said exterior surface and said interior surface.

5. The protective device of claim 1, wherein each said side portion includes a side member and a base element, with said side members being connected to said central member and said base element of each said side portion being connected to a respective said side member distal from said central member.

6. The protective device of claim 5, wherein said base elements are generally planar and extend outwardly relative to said side members.

7. The protective device of claim 6, wherein said central member is generally planar, and wherein said base elements and said central member are generally parallel.

8. The protective device of claim 1, wherein said central member and said side portions are unitarily constructed from foam.

9. The protective device of claim 8, wherein said central member and said side portions are pliable with said side members being sufficiently rigid to support said central member in a freestanding orientation.

10. The protective device of claim 1, further including a retention member operable to releasably secure objects to said protective device.

11. The protective device of claim 10, wherein said retention member comprises a hook and loop fastener strap.

12. A protective device for protecting the face and/or head of an individual receiving medical treatment, said protective device comprising:
a central member having an exterior surface and an interior surface with an aperture extending between said exterior surface and said interior surface; and
a pair of opposed side members affixed to said central member and extending downwardly there from relative to a plane defined by said central member, each said side member having a base element connected thereto distally from said central member;
said central member and said side members defining a cavity and an opening to said cavity with said protective device configured to being positioned over the head of an individual receiving medical treatment in a supine position with said base elements being supported on a support surface adjacent the individual's head and said cavity being disposed over the individual's head with the individual's neck extending into said opening and said side members supporting said central member adjacent the individual's face and said aperture exposing the individual's eyes, nose, and/or mouth.

13. The protective device of claim 12, wherein said aperture comprises a first aperture and said central member further includes a second aperture extending between said exterior surface and said interior surface.

14. The protective device of claim 12, wherein said base elements are generally planar and extend outwardly relative to said side members, said central member is generally planar, and said base elements and said central member are generally parallel.

15. The protective device of claim 12, wherein said central member, said side members and said base elements are unitarily constructed of a pliable foam with said side members being sufficiently rigid to support said central member in a freestanding orientation.

16. The protective device of claim 12, further including a retention member operable to releasably secure objects to said protective device.

17. A method of protecting the face and/or head of an individual receiving medical treatment, said method comprising:
providing a protective device, said protective device including a central member and a pair of opposed side portions affixed to said central member and extending downwardly there from relative to a plane defined by said central member, said central member having an exterior surface and an interior surface with said central member and said side portions defining a cavity and an opening to said cavity;
said providing said protective device for positioning about the head of an individual receiving medical treatment in a supine position with said side portions being supported on a support surface adjacent the individual's head and said cavity being disposed over the individual's head with the individual's neck extending into said opening and said interior surface being aligned with the individual's face.

18. The method of claim 17, wherein said central member includes an aperture extending between said exterior surface and said interior surface, and wherein said aperture exposes an individual's eyes, nose, and/or mouth when said protective device is positioned about the individual's head.

19. The method of claim 17, wherein each said side portion includes a side member and a base element, with said side members being connected to said central member and said base element of each said side portion being connected to a respective said side member distal from said central member, and wherein said base elements are generally planar and extend outwardly relative to said side members.

20. The method of claim 17, wherein said central member and said side portions are unitarily constructed from foam, and wherein said central member and said side portions are pliable with said side members being sufficiently rigid to support said central member in a freestanding orientation.
